(19) United States
(12) Patent Application Publication
(10) Pub. No.: US 2026/0316066 A1
(43) Pub. Date: Oct. 15, 2026

(54) MWW-STRUCTURED MOLECULAR SIEVE, PREPARATION METHOD THEREFOR AND USE THEREOF

(71) Applicant: PetroChina Company Limited, Beijing (CN)

(72) Inventors: Yan Cui, Beijing (CN); Shiyong Xing, Beijing (CN); Minghan Han, Beijing (CN); Xiaohua Wang, Beijing (CN); Chengyu Guo, Beijing (CN); Yin Xie, Beijing (CN); Hongyue Yu, Beijing (CN); Liang Qiao, Beijing (CN); Kebin Chi, Beijing (CN); Shang Zhang, Beijing (CN)

(21) Appl. No.: 19/108,603

(22) PCT Filed: Dec. 13, 2022
(86) PCT No.: PCT/CN2022/138658

§ 371 Date: Mar. 4, 2025

(30) Foreign Application Priority Data
Nov. 8, 2022 (CN) 202211395051.X

(51) Int. Cl. B01J 37/00; B01J 29/70; B01J 35/61; B01J 35/63; B01J 37/03; B01J 37/04; C01B 39/48; C07C 2/66

(52) U.S. Cl. CPC B01J 37/0018; B01J 29/7038; B01J 35/615; B01J 35/617; B01J 35/618; B01J 35/635; B01J 35/638; B01J 37/036; B01J 37/04; C01B 39/48; C07C 2/66; C01P 2002/72; C01P 2004/03; C01P 2004/04; C01P 2006/12; C01P 2006/14; C07C 2529/89

(57) ABSTRACT

An MWW-structured molecular sieve, a preparation method therefor and a use thereof. The method comprises mixing an aluminum source, water, an alkali source, a first template agent, a second template agent, a silicon source, and a seed crystal, and crystallizing the mixture to obtain the MWW-structured molecular sieve, wherein the first template agent comprises cyclohexylamine, and the second template agent comprises diisopropylamine, dibutylamine, diisobutylamine, 1,4-azabicyclo[2.2.2]octane, 1,6-hexamethylenediamine, and N,N,N,N-tetramethyl-1,6-hexamethylenediamine.

### TECHNICAL FIELD

The present disclosure relates to the technical field of molecular sieve material synthesis, and in particular to an MWW-structured molecular sieve and the preparation method and use thereof.

### BACKGROUND

MWW-structured molecular sieves have a very typical two-dimensional lamellar morphology, with a lamellar structure consisting of a number of base monolayers stacked together, which also forms unique pore structures, mainly including two-dimensional sinusoidal ten-membered ring apertures in the ab-axis plane and twelve-membered ring supercage and semi-supercage structures in the c-axis direction. The ten-membered ring apertures have an opening size of 0.41 nm×0.51 nm and are distributed outside the supercage structures; a twelve-membered ring supercage having a size of 0.71 nm×0.71 nm×1.82 nm is connected to six identical supercages in the surrounding ab-axis planes through ten-membered ring; the semi-supercages distributed on the surface of a lamellar layer have a size of 0.71 nm×0.71 nm×0.8 nm, and have open twelve-membered openings that are very favorable for the diffusion of reactants. MWW-structured molecular sieves have excellent catalytic reaction performance for macromolecular reactions, and have been widely used in alkylation and other reaction processes.

MWW-structured molecular sieves can be divided into many types according to the different manners in which the base monolayers are stacked, mainly including MCM-22 molecular sieves, MCM-49 molecular sieves having an oxygen bridge between monolayers, MCM-36 molecular sieves having a column support structure between layers, MCM-56 molecular sieves having disordered interlayer stacking, ITQ-2 molecular sieve having a single-layer structure, and the like. Among them, MCM-22 molecular sieves are obtained after dehydration of silicon hydroxyl groups between the layers to form an oxygen bridge upon roasting, and the crystal structure of the roasted MCM-22 molecular sieves is the same as that of MCM-49 molecular sieves.

The template agents used for directly inducing the synthesis of MWW-structured molecular sieves generally include hexamethyleneimine, piperidine, and homopiperazine. Among them, hexamethyleneimine is most widely used, but has the disadvantages of volatility, flammability and high toxicity. Piperidine is rarely commercially available. Homopiperazine is expensive and difficult to use. Therefore, it is very important to develop an inexpensive and green method for synthesizing MWW-structured molecular sieves.

The existing synthesis of MWW-structured molecular sieves is usually achieved by using hexamethyleneimine together with another organic amine for the synthesis of molecular sieves such as MCM-22.

As seen from the prior art, the synthesis of MWW-structure molecular sieves cannot be realized directly with a crystal seed due to the special characteristics thereof, and complete MCM-49 molecular sieves can be constructed by adding an organic amine on the basis of the addition of a crystal seed. To date, there is no precedent of synthesizing MCM-22 molecular sieves without using hexamethyleneimine, piperidine or homopiperazine, and thus there is an urgent need to develop a highly operable, environmentally friendly and inexpensive method for the preparation of MWW molecular sieves such as MCM-22 molecular sieves.

### SUMMARY

In order to solve the above problems, an object of the present disclosure is to provide an MWW-structured molecular sieve and the preparation method and use thereof. In the present disclosure, the MWW-structured molecular sieve can be directly synthesized in a crystallization process that is synergistically assisted by a crystal seed and two template agents, without the use of hexamethyleneimine, piperidine, or homopiperazine.

In order to achieve the above object, the present disclosure provides a preparation method of an MWW-structured molecular sieve, comprising: mixing an aluminum source, water, an alkali source, a first template agent, a second template agent, a silicon source, and a crystal seed to form a gel, and subjecting the gel to crystallization, to obtain the MWW-structured molecular sieve; wherein the first template agent comprises cyclohexylamine, and the second template agent may comprise one or more of diisopropylamine (with a molecular formula of C₆H₁₅N), di-n-butylamine (with a molecular formula of C₈H₁₉N), diisobutylamine (with a molecular formula of C₈H₁₉N), 1,4-diazabicyclo[2.2.2]octane (with a molecular formula of C₆H₁₂N₂), 1,6-hexanediamine (with a molecular formula of C₆H₁₆N₂), N,N,N,N-tetramethyl-1,6-hexanediamine (CAS No. 111-18-2, with a molecular formula of C₁₀H₂₄N₂).

In the above preparation method, the first template agent and the second template agent used are both low-toxic and inexpensive organic amines. Among them, the first template agent is used as the primary template agent, which can cooperate with the crystal seed to realize the construction of the molecular sieve base layer structure. The second template agent is used as a secondary template agent, and an aliphatic amine with a carbon number of 6 to 10 and a N atom with sp³ hybridization is used as the second template agent, which can form interlayer hydrogen bonds with the silicon hydroxyl groups on the surface of the monolayer structure owing to the empty orbital of the sp³ hybridized orbitals of the nitrogen atom, while the hydrogen bond-forming process does not affect the overall crystallization result of the molecular sieve. Thus, in some embodiments, the MWW-structured molecular sieve obtained by the preparation method described above has interlayer hydrogen bonds formed from silicon hydroxyl groups and the second template agent.

It has been found by research of the present disclosure that it is not possible to obtain an MWW-structured molecular sieve having interlayer hydrogen bonds by using only one of the first template agent, the second template agent, or the crystal seed described above. For example, in the case of omitting the second template agent, using only the first template agent and the crystal seed can only produce a MCM-49 molecular sieve, which has interlayer oxygen bridge bonds rather than hydrogen bonds. The above preparation method provided by the present disclosure allows for the synthesis of an MWW-structured molecular sieve having interlayer hydrogen bonds by a synergistic effect of the first template agent, the second template agent, and the crystal seed, without using hexamethyleneimine, piperidine or homopiperazine. In some specific embodiments, the molar ratio of the first template agent to the second template agent is generally controlled to be 0.5-20:1, for example, it can be controlled to be 0.5-1.5:1.

In specific embodiments of the present disclosure, with the alkali source denoted as MOH (M is the cation-forming atom in the alkali source, and the valence state of M is generally univalent; for example, if the alkali source is NaOH, M is Na), the silicon source in terms of SiO₂, the aluminum source in terms of Al₂O₃, the alkali source in terms of M₂O, the sum of the first template agent and the second template agent is denoted as T, the chemical composition of the gel generally satisfies the following molar ratios: Al₂O₃/SiO₂=0.005-0.05, M₂O/SiO₂=0.03-0.50, T/SiO₂=0.10-0.75, H₂O/SiO₂=8-120; and for the crystal seed in terms of dry mass, the mass ratio of the crystal seed to the silicon source generally satisfies: crystal seed/SiO₂=0.01-0.25.

In some specific embodiments, the molar ratio of the silicon source to the aluminum source may satisfy: Al₂O₃/SiO₂=0.01-0.05, and may further satisfy Al₂O₃/SiO₂=0.01-0.02.

In some specific embodiments, the molar ratio of the silicon source to water may satisfy: H₂O/SiO₂=15-120, or alternatively may satisfy H₂O/SiO₂=8-13.

In specific embodiments of the present disclosure, the temperature of the crystallization is generally controlled to be 120-170° C., e.g., 120° C., 125° C., 130° C., 135° C., 140° C., 145° C., 150° C., 155° C., 160° C., 165° C., 170° C. The duration of the crystallization is generally controlled to be 12-120 h, e.g., 12 h, 20 h, 30 h, 40 h, 50 h, 60 h, 70 h, 72 h, 80 h, 90 h, 100 h, 110 h, 120 h.

In specific embodiments of the present disclosure, the component of the silicon source may include silica, silicate salt, silicate ester and the like. Specifically, the silicon source may include one or more of silica sol, solid silica gel, white carbon black, water glass, ethyl orthosilicate and the like.

In specific embodiments of the present disclosure, the aluminum source may include one or more of meta-aluminate (e.g., sodium meta-aluminate), aluminum sulfate, alumina, pseudo boehmite and the like.

In specific embodiments of the present disclosure, the crystal seed is generally a molecular sieve having an MWW topology. For example, commercially available MCM-22 molecular sieves, MCM-49 molecular sieves and the like may be used. In some specific embodiments, a molecular sieve having an MWW topology obtained by the preparation method of the present disclosure may also be used as the crystal seed. The crystal seed is preferably a MCM-22 molecular sieve without template agent removal, which has interlayer hydrogen bonds in the molecular sieve, and when it is used as a crystal seed in the above preparation process, it is favorable for obtaining a molecular sieve with an MWW structure having interlayer hydrogen bonds which is similar to that of the MCM-22 molecular sieve.

In specific embodiments of the present disclosure, the alkali source may comprise sodium hydroxide and/or potassium hydroxide and the like.

In specific embodiments of the present disclosure, the above preparation method comprises: mixing an aluminum source, water, an alkali source, a first template agent, and a second template agent to obtain an intermediate solution, adding a silicon source and a crystal seed to the intermediate solution and mixing them to form a gel, and subjecting the gel to crystallization, to obtain the MWW-structured molecular sieve. Synthesizing in the above order facilitates uniformly dispersing each raw material and promoting the crystallization reaction. In some specific embodiments, the first template agent and the second template agent are added after the aluminum source, which facilitates the aluminum source to be completely dissolved and uniformly dispersed in the solution. The addition of the silicon source may be slow, and the crystal seed may be added after the silicon source, which can improve the homogeneity and stability of the reaction system, and avoid the problem of forming a colloid with the aluminum source after rapidly adding the silicon source and causing a non-uniform dispersion of the reaction system.

The present disclosure also provides an MWW-structured molecular sieve obtained by the above preparation method. The MWW-structured molecular sieve provided by the present disclosure has an MWW topology and interlayer hydrogen bonds. In some specific embodiments, in an XRD pattern of the MWW-structured molecular sieve, prominent characteristic peaks are present at approximately 6.5-7.15° (e.g., 7.09°) and 7.18°, respectively.

The MWW-structured molecular sieve of the present disclosure has interlayer hydrogen bonds and are similar in structure to that of a MCM-22 molecular sieve which has not been roasted (and also with the template agent not removed), and it can thus be regarded as a precursor of the MCM-22 molecular sieve (which can be designated as a MCM-22(P) molecular sieve). The MWW-structured molecular sieve of the present disclosure has good potential for modification. For example, it can be prepared into a MCM-36 molecular sieve after swelling and column bracing, or into an ITQ-2 molecular sieve after swelling and delamination. It has been found by research of the present disclosure that an MCM-49 molecular sieve has no interlayer hydrogen bond and could not be swelled and delaminated; both an MCM-22 molecular sieve produced with a conventional template agent (hexamethyleneimine) and the MWW-structured molecular sieve provided by the present disclosure have the capacity for swelling and delaminating because both of them have interlayer hydrogen bonds. Compared to an MCM-22 molecular sieve produced with a conventional template agent (hexamethylenimine), the MWW structure molecular sieve provided by the present disclosure has less difficulty in delamination, which can be demonstrated by a pH closer to neutral in an alkaline environment required for delamination. This indicates that the interlayer hydrogen bonding strength of the MWW structure molecular sieve provided by the present disclosure is more moderate than that of the MCM-22 molecular sieve, which is more favorable to retaining the integrity of the lamellar crystal structure of the molecular sieve, improving the stability of the molecular sieve, and decreasing the loss of the catalytic active sites when the molecular sieve is modified by column bracing or delamination, and is therefore advantageous in improving the catalytic activity and stability of the molecular sieve when it is applied in an alkylation reaction, an isomerization reaction or a cracking reaction.

In specific embodiments of the present disclosure, the MWW-structured molecular sieve has a high specific surface area which may be up to 500 m²·g or more.

In specific embodiments of the present disclosure, the MWW-structured molecular sieve may have a micropore specific surface area of up to 350 m²·g or more, and a mesopore specific surface area of up to 140 m²·g or more.

In specific embodiments of the present disclosure, the MWW-structured molecular sieve may have a pore volume of up to 0.60 cm³·g or more.

In specific embodiments of the present disclosure, the MWW-structured molecular sieve may have a micropore volume of up to 0.16 cm³·g or more.

The present disclosure also provides use of the MWW-structured molecular sieves in catalyzing an alkylation reaction, an isomerization reaction or a cracking reaction. The catalytic effect of the molecular sieve provided by the present disclosure is not lower than that of commercially available MCM-22 molecular sieves. In some specific embodiments, when the MWW-structured molecular sieve of the present disclosure is applied to an alkylation reaction, the olefin conversion can reach 99.97% or more, and the selectivity can reach 42% or more, or even 96% or more.

The advantageous effects of the present disclosure are as follows.

1) The present disclosure depends on the construction of a base layer structure with a crystal seed and a first template agent, and on the formation of interlayer hydrogen bonds with a second template agent, and without using traditional template agents such as hexamethyleneimine, piperidine and homopiperazine, and allows the product to have characteristics similar to those of MCM-22 molecular sieves and exhibit high catalytic capacity.

2) Both the first template agent and the second template agent used in the present disclosure are low-toxic, inexpensive and readily available organic amines, which can greatly improve the operability of the production of the MWW-structured molecular sieve, and is of great significance in practical application.

### BRIEF DESCRIPTION OF THE ACCOMPANYING DRAWINGS

FIG. 1 shows an XRD pattern of the crystal seed.

FIG. 2 shows an SEM photograph of the crystal seed.

FIG. 3 shows an XRD pattern of the molecular sieve synthesized in Example 1.

FIG. 4 shows an SEM photograph of the molecular sieve synthesized in Example 1.

FIG. 5 shows an XRD pattern of the molecular sieve synthesized in Example 2.

FIG. 6 shows an SEM photograph of the molecular sieve synthesized in Example 2.

FIG. 7 shows an XRD pattern of the molecular sieve synthesized in Comparative Example 1.

FIG. 8 shows an SEM photograph of the molecular sieve synthesized in Comparative Example 1.

FIG. 9 shows an XRD pattern of the molecular sieve synthesized in Comparative Example 2.

FIG. 10 shows an SEM photograph of the molecular sieve synthesized in Comparative Example 2.

FIG. 11 shows an XRD pattern of the molecular sieve synthesized in Comparative Example 3.

FIG. 12 shows an SEM photograph of the molecular sieve synthesized in Comparative Example 3.

FIG. 13 shows a TEM image of the molecular sieve synthesized in Comparative Example 1.

FIG. 14 shows a TEM image of the molecular sieve synthesized in Example 1.

### DETAILED DESCRIPTION

In order to have a clearer understanding of the technical features, purposes and beneficial effects of the present disclosure, the following detailed description of the technical program of the present disclosure is provided, but is not to be construed as a limitation to the implementable scope of the present disclosure.

The following examples of the present disclosure are described in detail: the following examples are implemented on the premise of the technical solutions of the present disclosure, and although detailed implementation methods and processes are given, the protection scope of the present disclosure is not limited to these examples. The experimental methods in the following examples in which specific conditions are not indicated are usually carried out in accordance with conventional conditions.

The raw materials used in the following Examples and Comparative Examples are:
Silicon source: silica sol (SiO₂ content: 40%; mass content, same hereinafter), solid silica gel (SiO₂ content: 95%), white carbon black (SiO₂ content; 93%), ethyl orthosilicate (SiO₂ content: 98%, in terms of elemental silicon content);
Aluminum source: sodium meta-aluminate (Al₂O₃ content: 41%, in terms of elemental aluminum content), aluminum sulfate (Al₂O₃ content: 15%, in terms of elemental aluminum content), alumina (Al₂O₃ content: 95%, in terms of elemental aluminum content), pseudo boehmite (Al₂O₃ content: 70%, in terms of elemental aluminum content);
Alkali source: sodium hydroxide (99%), potassium hydroxide (99%);
First template agent: cyclohexylamine (99%);
Second template agent: diisopropylamine (99%), di-n-butylamine (99%), diisobutylamine (99%), 1,4-diazabicyclo[2.2.2]octane (99%), 1,6-hexanediamine (99%), N,N,N,N-tetramethyl-1,6-hexanediamine (99%);
Crystal seed: MCM-22 molecular sieve from Mobil Co., which have not been subjected to a roasting process to remove template agents;
Water: deionized water.

The molecular sieve products of the Examples and Comparative Examples used for characterization were first subjected to a roasting process at 540° C. in an air atmosphere, followed by BET characterization.

### Example 1

This example provides an MWW-structured molecule, of which the preparation method comprises:

1) 1.87 g of sodium hydroxide was added to 120 g of deionized water and dissolved with stirring; 2.15 g of sodium meta-aluminate was added and dissolved with continuous and vigorous stirring for 1 h; 9.50 g of cyclohexylamine was added slowly with continuous and vigorous stirring for 0.5 h; and 6.50 g of diisopropylamine was added slowly with continuous and vigorous stirring for 0.5 h, to obtain an intermediate solution.

2) 80 g of silica sol was added slowly to the intermediate solution with continuous and vigorous stirring for 3 h; a crystal seed having a mass of 3% of the silica content in the silica sol was added with continuous and vigorous stirring for 1 h to obtain a gel. The gel was crystallized at 140° C. for 60 h. At the end of crystallization, the temperature was reduced to room temperature. The crystallization product was washed and filtered with deionized water and dried at 120° C. for 4 h, to obtain a molecular sieve product.

In the above experiment, with the content of the silicon source in terms of SiO₂, and the content of the aluminum source in terms of Al₂O₃, the molar ratio of the silicon source to the aluminum source was: SiO₂/Al₂O₃=62.

### Example 2

This example provides an MWW-structured molecule, of which the preparation method comprises:

1) 1.87 g of sodium hydroxide was added to 120 g of deionized water and dissolved with stirring; 2.50 g of sodium meta-aluminate was added and dissolved with continuous and vigorous stirring for 1 h; 9.00 g of cyclohexylamine was added slowly with continuous and vigorous stirring for 0.5 h; and 8.00 g of 1,6-hexanediamine was added slowly with continuous and vigorous stirring for 0.5 h, to obtain an intermediate solution.

2) 80 g of silica sol was added slowly to the intermediate solution with continuous and vigorous stirring for 3 h; a crystal seed having a mass of 5% of the silica content in the silica sol was added with continuous and vigorous stirring for 1 h to obtain a gel. The gel was crystallized at 145° C. for 72 h. At the end of crystallization, the temperature was reduced to room temperature. The crystallization product was washed and filtered with deionized water, and dried at 120° C. for 4 h, to obtain a molecular sieve product.

In the above experiment, with the content of the silicon source in terms of SiO₂, and the content of the aluminum source in terms of Al₂O₃, the molar ratio of the silicon source to the aluminum source was: SiO₂/Al₂O₃=53.3.

### Example 3

This example provides an MWW-structured molecule, of which the preparation method comprises:

1) 1.95 g of sodium hydroxide was added to 130 g of deionized water and dissolved with stirring; 2.33 g of sodium meta-aluminate was added and dissolved with continuous and vigorous stirring for 1 h; 9.00 g of cyclohexylamine was added slowly with continuous and vigorous stirring for 0.5 h; and 9.00 g of 1,4-diazabicyclo[2.2.2]octane was added slowly with continuous and vigorous stirring for 0.5 h, to obtain an intermediate solution.

2) 80 g of silica sol was added slowly to the intermediate solution with continuous and vigorous stirring for 3 h; a crystal seed having a mass of 5% of the silica content in the silica sol was added with continuous and vigorous stirring for 1 h to obtain a gel. The gel was crystallized at 140° C. for 72 h. At the end of crystallization, the temperature was reduced to room temperature. The crystallization product was washed and filtered with deionized water, and dried naturally for 48 h, to obtain a molecular sieve product.

In the above experiment, with the content of the silicon source in terms of SiO₂, and the content of the aluminum source in terms of Al₂O₃, the molar ratio of the silicon source to the aluminum source was: SiO₂/Al₂O₃=57.2.

### Example 4

This example provides an MWW-structured molecule, of which the preparation method comprises:

1) 1.93 g of sodium hydroxide was added to 150 g of deionized water and dissolved with stirring; 1.50 g of sodium meta-aluminate was added and dissolved with continuous and vigorous stirring for 1 h; 12.25 g of cyclohexylamine was added slowly with continuous and vigorous stirring for 0.5 h; and 8.50 g of N,N,N,N-tetramethyl-1,6-hexanediamine was added slowly with continuous and vigorous stirring for 0.5 h, to obtain an intermediate solution.

2) 80 g of silica sol was added slowly to the intermediate solution with continuous and vigorous stirring for 3 h; a crystal seed having a mass of 10% of the silica content in the silica sol was added with continuous and vigorous stirring for 1 h to obtain a gel. The gel was crystallized at 140° C. for 100 h. At the end of crystallization, the temperature was reduced to room temperature. The crystallization product was washed and filtered with deionized water, and dried at 120° C. for 4 h, to obtain a molecular sieve product.

In the above experiment, with the content of the silicon source in terms of SiO₂, the content of the aluminum source in terms of Al₂O₃, the molar ratio of the silicon source to the aluminum source was: SiO₂/Al₂O₃=88.9.

### Example 5

This example provides an MWW-structured molecule, of which the preparation method comprises:

1) 2.21 g of sodium hydroxide was added to 175 g of deionized water and dissolved with stirring; 1.25 g of sodium meta-aluminate was added and dissolved with continuous and vigorous stirring for 1 h; 7.55 g of cyclohexylamine was added slowly with continuous and vigorous stirring for 0.5 h; and 10.50 g of diisobutylamine was added slowly with continuous and vigorous stirring for 0.5 h, to obtain an intermediate solution.

2) 80 g of silica sol was added slowly to the intermediate solution with continuous and vigorous stirring for 3 h; a crystal seed having a mass of 15% of the silica content in the silica sol was added with continuous and vigorous stirring for 1 h to obtain a gel. The gel was crystallized at 140° C. for 120 h. At the end of crystallization, the temperature was reduced to room temperature. The crystallization product was washed and filtered with deionized water, and dried naturally for 48 h, to obtain a molecular sieve product.

In the above experiment, with the content of the silicon source in terms of SiO₂, the content of the aluminum source in terms of Al₂O₃, the molar ratio of the silicon source to the aluminum source was: SiO₂/Al₂O₃=106.6.

### Comparative Example 1

This comparative example provides an MWW-structured molecular sieve, of which the preparation method comprises:

1) 1.95 g of sodium hydroxide was added to 130 g of deionized water and dissolved with stirring; 2.33 g of sodium meta-aluminate was added with continuous and vigorous stirring for 1 h; 9.00 g of cyclohexylamine was added slowly with continuous and vigorous stirring for 0.5 h, to obtain an intermediate solution.

2) 80 g of silica sol was added slowly to the intermediate solution with continuous and vigorous stirring for 3 h; a crystal seed having a mass of 5% of the silica content in the silica sol was added with continuous and vigorous stirring for 1 h to obtain a gel. The gel was crystallized at 140° C. for 72 h. At the end of crystallization, the temperature was reduced to room temperature. The crystallization product was washed and filtered with deionized water, and dried naturally for 48 h, to obtain a molecular sieve product.

### Comparative Example 2

This comparative example provides an MWW-structured molecular sieve, of which the preparation method comprises:

1) 1.87 g of sodium hydroxide was added to 120 g of deionized water and dissolved with stirring; 2.15 g of sodium meta-aluminate was added and dissolved with continuous and vigorous stirring for 1 h; 19.0 g of hexamethylenimine (the molar ratio of template agent to silicon source was 0.35:1) was added slowly with continuous and vigorous stirring for 0.5 h, to obtain an intermediate solution.

2) 80 g of silica sol was added slowly to the intermediate solution with continuous and vigorous stirring for 3 h, to obtain a crystallized gel. The crystallized gel was crystallized at 155° C. for 60 h. At the end of crystallization, the temperature was reduced to room temperature. The product was washed and filtered with deionized water, and dried at 120° C. for 4 h, to obtain a molecular sieve product.

The hexamethylenimine used in this method is a typical template agent for the preparation of MCM-22 molecular sieves. In this comparative example, although pure phase MCM-22 molecular sieves can be obtained, the template agent hexamethylenimine was used in a large amount, and was expensive and toxic.

### Comparative Example 3

This comparative example provides an MWW-structured molecular sieve, of which the preparation method comprises:

1) 1.87 g of sodium hydroxide was added to 120 g of deionized water and dissolved with stirring; 2.15 g of sodium meta-aluminate was added with continuous and vigorous stirring for 1 h; 9.50 g of cyclohexylamine and 9.50 g of hexamethylenimine were added slowly with continuous and vigorous stirring for 0.5 h, to obtain an intermediate solution.

2) 80 g of silica sol was added slowly to the intermediate solution with continuous and vigorous stirring for 3 h, to obtain a crystallized gel. The crystallized gel was crystallized at 155° C. for 60 h. At the end of crystallization, the temperature was reduced to room temperature. The product was washed and filtered with deionized water, and dried at 120° C. for 4 h, to obtain a molecular sieve product.

### Test Example 1

This test example was used to structurally characterize the above Examples and Comparative Examples.

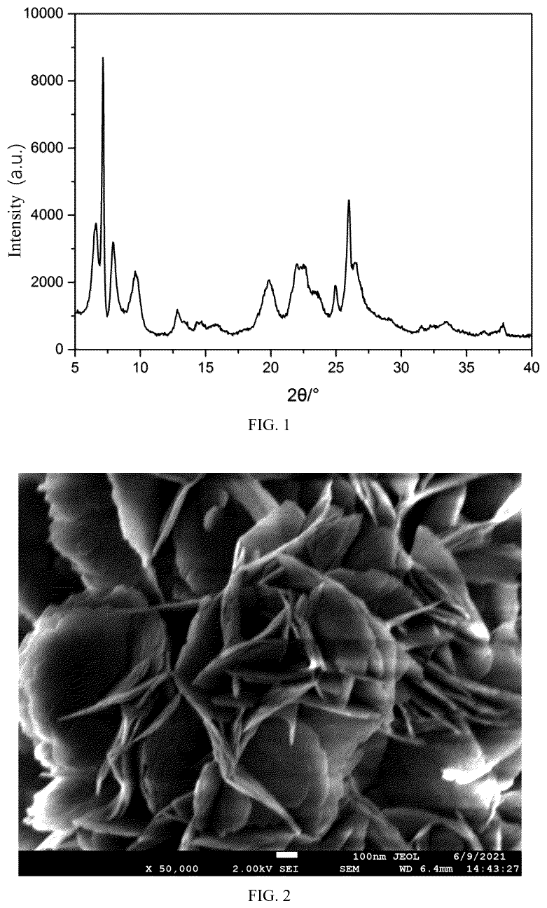

FIGS. 1 and 2 show the XRD pattern and SEM photograph of the crystal seed (MCM-22 molecular sieves sold by Mobil Co.) used in the above Examples and Comparative Examples, respectively.

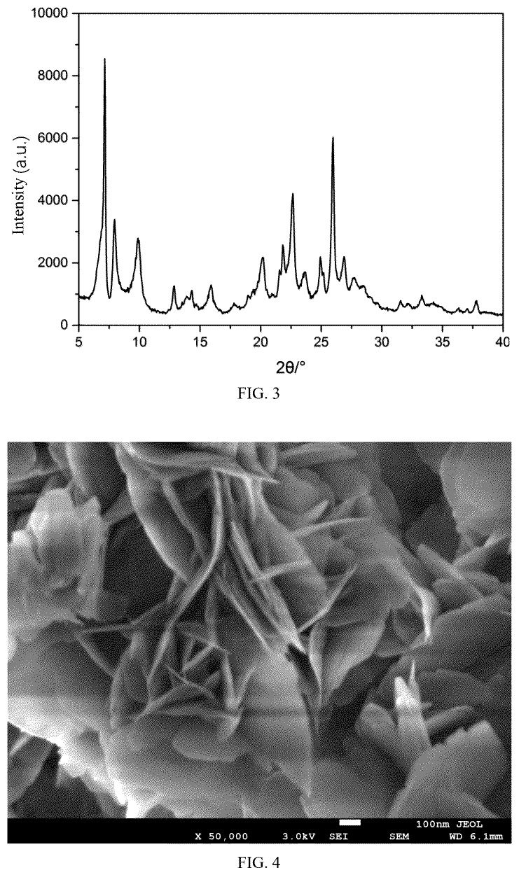

FIGS. 3 and 4 show the XRD pattern and SEM photograph of the molecular sieve product of Example 1, respectively. As can be seen from FIGS. 3 and 4, the molecular sieve product prepared in Example 1 has typical MWW-structural characteristic peaks, and the molecular sieve product has a morphology showing nanosheets stacked in the form of flower clusters.

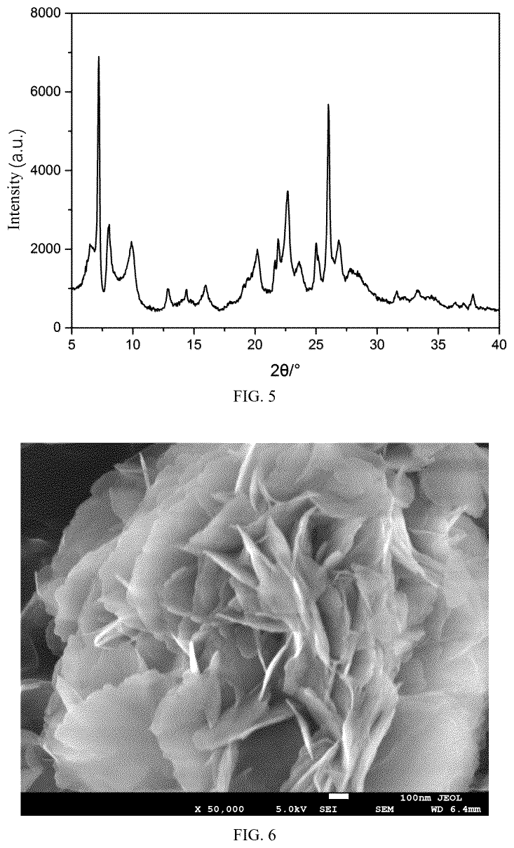

FIGS. 5 and 6 show the XRD pattern and SEM photograph of the molecular sieve product of Example 2, respectively. As can be seen from FIGS. 5 and 6, the molecular sieve product prepared in Example 2 has typical MWW-structural characteristic peaks, and the molecular sieve product has a morphology showing nanosheets stacked in the form of flower clusters.

XRD and SEM characterization of the molecular sieve products of Example 3, Example 4 and Example 5 also show that the above samples have typical MWW-structural characteristic peaks, and the molecular sieve products have a morphology showing nanosheets stacked in the form of flower clusters.

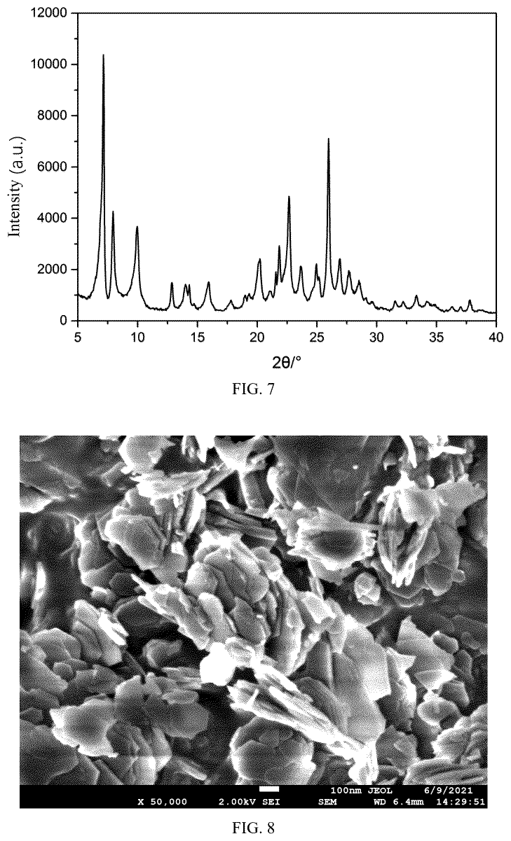

FIGS. 7 and 8 show the XRD pattern and SEM photograph of the molecular sieve product of Comparative Example 1, respectively. As can be seen from FIGS. 7 and 8, the molecular sieve product prepared in Comparative Example 1 has typical MWW-structural characteristic peaks and can be categorized into MCM-49 molecular sieves. Moreover, this molecular sieve product has a morphology showing orderly stacked nanosheets.

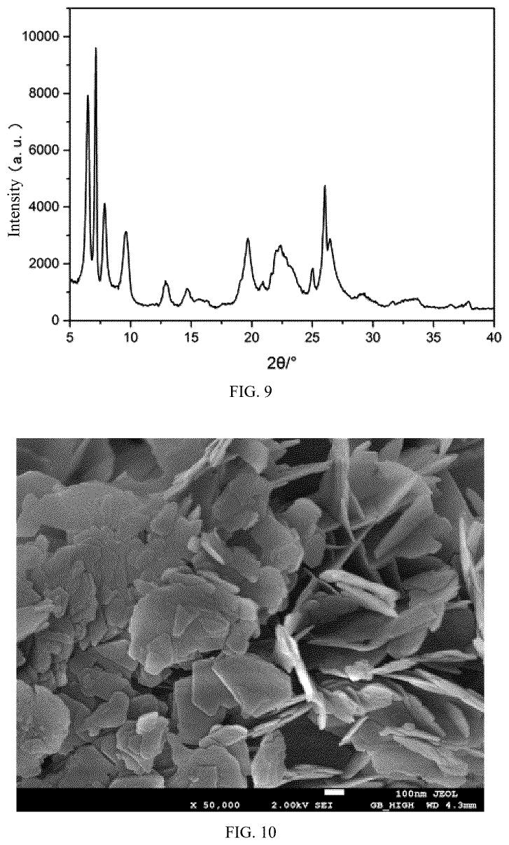

FIGS. 9 and 10 show the XRD pattern and SEM photograph of the molecular sieve product of Comparative Example 2, respectively. It can be seen that the molecular sieve product prepared in Comparative Example 2 has typical MWW-structural characteristic peaks and can be categorized into MCM-49 molecular sieves, and the molecular sieve has a morphology in the form of nanosheets.

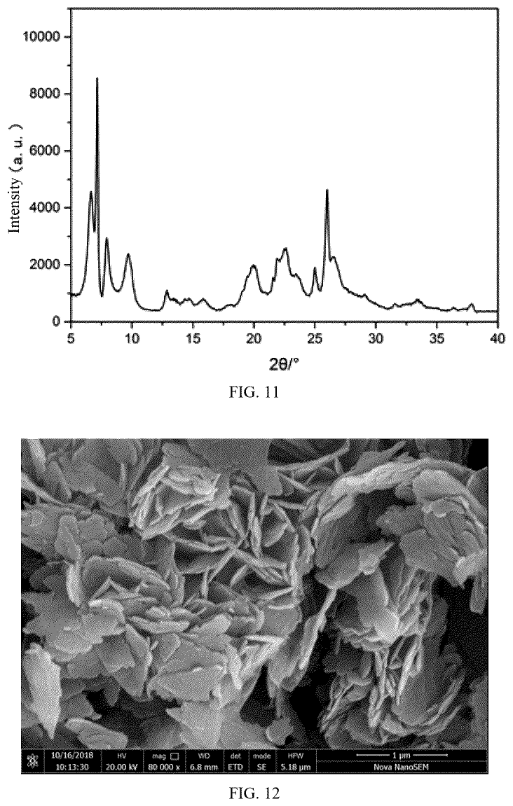

FIGS. 11 and 12 show the XRD pattern and SEM photograph of the molecular sieve product of Comparative Example 3, respectively. It can be seen that the molecular sieve product prepared in Comparative Example 3 has typical MWW-structural characteristic peaks and is a mixture of MCM-22 and MCM-49 molecular sieves, and the molecular sieve has a morphology in the form of stacked nanosheets.

Comparison of the XRD results of the molecular sieve products of Examples 1 to 5 with the XRD results of Comparative Example 1 shows that the molecular sieve products of Examples 1 to 5 have independent characteristic peaks at 7.09° (corresponding to the 002 crystal plane) and 7.18°, respectively. These two characteristic peaks indicate there is a distinct interlayer spacing between 002 crystal planes in this molecular sieve product, and this is because the second template agent induces interlayer hydrogen bonding between neighboring 002 crystal planes and creates a certain steric hindrance, enabling a distinct spacing between neighboring crystal planes. However, the XRD pattern of the product of Comparative Example 1 has just one distinctive characteristic peak at 7.18°, but no characteristic peak at 7.09°. This is because 002 crystal planes are stacked on top of each other, with no distinct interlayer spacing, in the product of Comparative Example 1, and condensed oxygen bridges are present in the interlayers in the product of Comparative Example 1.

The hexamethyleneimine used in Comparative Example 2 is a typical template agent for the preparation of MCM-22 molecular sieves, and the molecular sieve obtained in Comparative Example 2 is a pure phase MCM-22 molecular sieve. Comparison of the XRD results of the molecular sieve products of Examples 1 to 5 with the XRD results of Comparative Example 2 shows that that the molecular sieves of Examples 1 to 5 have a structure similar to that of the MCM-22 molecular sieve, which further confirms that the molecular sieves of the present application have interlayer hydrogen bonds as in MCM-22 molecular sieves.

The above results can demonstrate that the product obtained by using cyclohexylamine alone as a template agent is a MCM-49 molecular sieve; whereas, by using cyclohexylamine and the second template agent as template agents, an MWW-structured molecular sieve having interlayer hydrogen bonds can be prepared, and the molecular sieve has a structure similar to that of the MCM-22 molecular sieve produced by Mobil Co.

Comparison of the product composition of Comparative Example 3 with that of Example 1 shows that, although a pure phase MWW-structured molecular sieve can be synthesized using hexamethyleneimine and cyclohexylamine as a composite template agent, the product of Comparative Example 3 is a mixture of MCM-22 and MCM-49 molecular sieves, and no pure phase MCM-22 structured molecular sieve can be obtained. The reason why pure phase molecular sieves cannot be obtained with the composite template agent of hexamethyleneimine and cyclohexylamine lies in that when cyclohexylamine is substituted for a certain proportion of hexamethyleneimine, since cyclohexylamine does not have the ability to form interlayer hydrogen bond, and hexamethyleneimine at a lower concentration cannot form enough hydrogen bonds, instead of a single pure phase molecular sieve, only a mixture of two molecular sieves can be obtained.

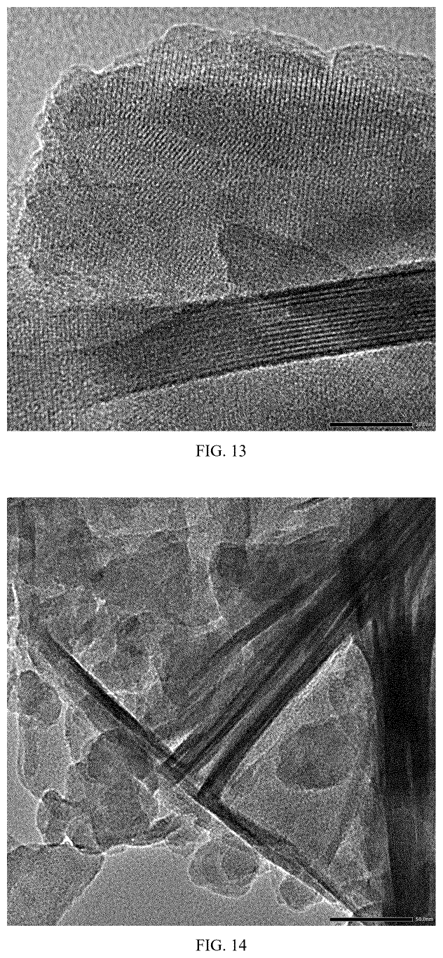

FIG. 13 shows a TEM image of the molecular sieve synthesized in Comparative Example 1, and FIG. 14 shows a TEM image of the molecular sieve synthesized in Example 1. Comparing FIGS. 13 and 14, it can be seen that the molecular sieve of Comparative Example 1 has distinctively stacked base layers with a layer thickness of about 20 nm. In contrast, the molecular sieve of Example 1 has a reduced number of stacked base layers, with a layer thickness of the molecular sieve of about 10 nm which is significantly lower than that of Comparative Example 1.

### Test Example 2

This test example provides the results of the specific surface area and pore volume of the molecular sieve products of Examples 1 to 5 and Comparative Example 1 measured by BET, as shown in Table 1, where S_{BET} is the specific surface area, S_{mic} is the micropore surface area, Sₑₓₜ is the mesopore surface area, Vₚₒᵣₑ is the pore volume, and V_{mic} is the micropore volume.

TABLE 1

| Sample | S_{BET}/m² · g | S_{mic}/m² · g | Sₑₓₜ/m² · g | Vₚₒᵣₑ/cm³ · g | V_{mic}/cm³ · g |
|---|---|---|---|---|---|
| Example 1 | 541.73 | 399.36 | 142.37 | 0.61 | 0.16 |
| Example 2 | 562.27 | 402.33 | 159.94 | 0.66 | 0.17 |
| Example 3 | 504.11 | 353.68 | 150.43 | 0.63 | 0.16 |
| Example 5 | 527.65 | 361.47 | 166.18 | 0.64 | 0.16 |
| Comparative | 419.42 | 362.87 | 56.55 | 0.52 | 0.16 |
| Example 1 | | | | | |
| Comparative | 469.32 | 351.33 | 117.99 | 0.53 | 0.16 |
| Example 2 | | | | | |
| Comparative | 451.85 | 346.87 | 104.98 | 0.53 | 0.17 |
| Example 3 | | | | | |

As can be seen from Table 1, the molecular sieves prepared in Examples 1 to 5 of the present disclosure have a greater microporous specific surface area, mesoporous specific surface area, total specific surface area and total pore volume than the molecular sieve products prepared in the Comparative Example 1 to 3, and the molecular sieves prepared in Examples 1 to 5 have a microporous volume not lower than the molecular sieve products prepared in the Comparative Example 1 to 3.

Combining the data of Table 1 with the results of FIGS. 13 and 14, it can be seen that in the preparation method provided by the present disclosure, by adding the second template agent, an empty sp³ orbital of the N atom in the second template agent can be utilized to form a hydrogen bond with the hydrogen in the silicon hydroxyl group. The molecular sieve thus obtained not only has an MWW topology, but also a molecular sieve structure similar to that of an MCM-22 molecular sieve, both having interlayer hydrogen bonds, and therefore has the properties of the MCM-22 molecular sieve. Moreover, the total specific surface area and total pore volume of the molecular sieves of the present disclosure are significantly increased. In addition, the molecular sieve products prepared by the present disclosure have a lower number of stacks and a smaller layer thickness, and the stacking morphology of the molecular sieves is altered compared to an MCM-49 molecular sieve.

Combining the results of Test Example 1 and Test Example 2, it can be seen that in Comparative Example 1 a pure phase MWW-structured molecular sieve with high crystallinity that can be categorized into an MCM-49 molecular sieve can be directly synthesized when cyclohexylamine is used as a template agent. However, since cyclohexylamine is unable to form interlayer hydrogen bonds, cyclohexylamine as a template agent is only suitable for the synthesis of MCM-49 molecular sieves, and not for the synthesis of other kinds of MWW-structured molecular sieves. In Comparative Example 3 in which a complex template agent was used by replacing a part of hexamethyleneimine with cyclohexylamine, it was not possible to form enough interlayer hydrogen bonds, and therefore only a mixture of MCM-22 molecular sieves and MCM-49 molecular sieves could be prepared, but not pure phase molecular sieves with a MCM-22 structure.

### Example 6

This example provides the use of the molecular sieve product of Example 1 in a catalytic alkylation reaction.

The molecular sieve product of Example 1 was taken and roasted at 540° C. under air atmosphere for 5 h to remove the template agent. It was then subjected to ammonium exchange in a 1 mol/L ammonium nitrate solution at 80° C. for 2 h and roasted again at 540° C. for 4 h under air atmosphere to obtain H-type molecular sieves.

90 g of the above H-type molecular sieves were mixed evenly with 15 g of pseudo boehmite, 55 g of nitric acid solution was added gradually while mixing and kneading, and then extruded into a φ2.0 mm cylindrical catalyst, which was cut into a cylindrical catalyst having a length of 2.5 mm. The catalyst was naturally dried at room temperature for 24 h, and then roasted at 550° C. for 6 h to obtain a finished catalyst.

2 g of the above finished catalyst was loaded into a fixed-bed reactor and a mixture of benzene and ethylene was introduced into the reactor. The reaction conditions were as follows: a ratio of benzene to alkene of 5, a reaction temperature of 330° C., a reaction pressure of 1.4 MPa, and a weight hourly space velocity of 2.0 h⁻¹.

The reaction resulted in a 99.99% alkene conversion and a 96.21% ethylbenzene selectivity after reacting for 500 h.

### Example 7

This example provides the use of the molecular sieve product of Example 2 in a catalytic alkylation reaction.

The molecular sieve product of Example 2 was taken and roasted at 540° C. under air atmosphere for 5 h to remove the template agent. It was then subjected to ammonium exchange in a 1 mol/L ammonium nitrate solution at 80° C. for 2 h and roasted again at 540° C. for 4 h under air atmosphere to obtain H-type molecular sieves.

90 g of the above H-type molecular sieves were mixed evenly with 15 g of pseudo boehmite, 55 g of nitric acid solution was added gradually while mixing and kneading, and then extruded into a φ2.0 mm cylindrical catalyst, which was cut into a cylindrical catalyst having a length of 2.5 mm. The catalyst was naturally dried at room temperature for 24 h, and then roasted at 550° C. for 6 h to obtain a finished catalyst.

2 g of the above finished catalyst was loaded into a fixed-bed reactor and a mixture of benzene and propylene was introduced into the reactor. The reaction conditions were as follows: a ratio of benzene to alkene of 4, a reaction temperature of 150° C., a reaction pressure of 2.5 MPa, and a weight hourly space velocity of 3.0 h⁻¹.

The reaction resulted in a 99.97% alkene conversion and a 99.20% isopropylbenzene selectivity after reacting for 300 h.

### Example 8

This example provides the use of the molecular sieve product of Example 3 in a catalytic alkylation reaction.

The molecular sieve product of Example 3 was taken and roasted at 540° C. under air atmosphere for 5 h to remove the template agent. It was then subjected to ammonium exchange in a 1 mol/L ammonium nitrate solution at 80° C. for 2 h and roasted again at 540° C. for 4 h under air atmosphere to obtain H-type molecular sieves.

90 g of the above H-type molecular sieves were mixed evenly with 15 g of pseudo boehmite, 55 g of nitric acid solution was added gradually while mixing and kneading, and then extruded into a φ2.0 mm cylindrical catalyst, which was truncated into a cylindrical catalyst having a length of 2.5 mm. The catalyst was naturally dried at room temperature for 24 h, and then roasted at 550° C. for 6 h to obtain a finished catalyst.

2 g of the above finished catalyst was loaded into a fixed-bed reactor and a mixture of benzene and n-dodecene was introduced into the reactor. The reaction conditions were as follows: a ratio of benzene to alkene of 15, a reaction temperature of 150° C., a reaction pressure of 3.0 MPa, and a weight hourly space velocity of 2.0 h⁻¹.

The reaction resulted in a 99.97% alkene conversion and a 42.01% 2-alkylbenzene selectivity after reacting for 300 h.

### Comparative Example 4

This example provides the use of the molecular sieve product of Comparative Example 1 in a catalytic alkylation reaction.

The molecular sieve product of Comparative Example 1 was taken and roasted at 540° C. under air atmosphere for 5 h to remove the template agent. It was then subjected to ammonium exchange in a 1 mol/L ammonium nitrate solution at 80° C. for 2 h and roasted again at 540° C. for 4 h under air atmosphere to obtain H-type molecular sieves.

90 g of the above H-type molecular sieves were mixed evenly with 15 g of pseudo boehmite, 55 g of nitric acid solution was added gradually while mixing and kneading, and then extruded into a φ2.0 mm cylindrical catalyst, which was cut into a cylindrical catalyst having a length of 2.5 mm. The catalyst was naturally dried at room temperature for 24 h, and then roasted at 550° C. for 6 h to obtain a finished catalyst.

2 g of the above finished catalyst was loaded into a fixed-bed reactor and a mixture of benzene and n-dodecene was introduced into the reactor. The reaction conditions were as follows: a ratio of benzene to alkene of 15, a reaction temperature of 150° C., a reaction pressure of 3.0 MPa, and a weight hourly space velocity of 2.0 h⁻¹.

The reaction resulted in a 76.32% alkene conversion and a 45.03% 2-alkylbenzene selectivity after reacting for 100 h.

Comparison of the results of Comparative Examples 4 with those of Examples 6 to 8 shows that the catalysts in Examples 6 to 8 have higher alkene conversion and isopropylbenzene selectivity. The reasons are as follows: the MCM-49 molecular sieve prepared in Comparative Example 1 does not have interlayer hydrogen bond, and the monolayers tend to stack on top of each other to form condensed oxygen bridge and have a high degree of stacking; in contrast, the molecular sieves prepared in each of the Examples have interlayer hydrogen bonds, and the hydrogen bonds as well as the template agent used to form the hydrogen bonds have a steric effect, which prevents the stacking of the layers of the molecular sieves and thus allows a low degree of stacking in the molecular sieves of the Examples; since the degree of stacking of the molecular sieves of the Examples is lower than the degree of stacking of the molecular sieve of Comparative Example 1, the layer thickness of the molecular sieves prepared in Examples 1 to 3 is smaller than that of the MCM-49 molecular sieve of Comparative Example 1, and higher number of surface catalytic active sites are exposed per unit mass of catalyst, which not only results in higher catalytic activity, but also avoids the problem of deactivation due to clogging during the reaction and extends the catalyst lifetime.

Seen from above, the preparation method provided by the present disclosure allows synthesizing an MWW-structured molecular sieve having interlayer hydrogen bonds without using hexamethyleneimine, piperidine or homopiperazine. This molecular sieve has properties similar to that of a MCM-22 molecular sieve and has high catalytic activity.

1. A preparation method of an MWW-structured molecular sieve, comprising:
mixing an aluminum source, water, an alkali source, a first template agent, a second template agent, a silicon source, and a crystal seed to form a gel, and subjecting the gel to crystallization to obtain the MWW-structured molecular sieve;
wherein the first template agent comprises cyclohexylamine, and the second template agent comprises one or more of diisopropylamine, di-n-butylamine, diisobutylamine, 1,4-diazabicyclo[2.2.2]octane, 1,6-hexanediamine, N,N,N,N-tetramethyl-1,6-hexanediamine.

2. The preparation method according to claim 1, wherein the molar ratio of the first template agent to the second template agent is 0.5-20:1.

3. The preparation method according to claim 1, wherein with the alkali source denoted as MOH, the silicon source in terms of SiO₂, the aluminum source in terms of Al₂O₃, the alkali source in terms of M₂O, and the sum of the first template agent and the second template agent denoted as T, the chemical composition of the gel satisfies the following molar ratios:
Al₂O₃/SiO₂=0.005-0.05, M₂O/SiO₂=0.03-0.50, T/SiO₂=0.10-0.75, H₂O/SiO₂=8-120;
and for the crystal seed in terms of dry mass, the mass ratio of the crystal seed to the silicon source satisfies: crystal seed/SiO₂=0.01-0.25.

4. The preparation method according to claim 3, wherein the chemical composition of the gel satisfies the following range of molar ratio: H₂O/SiO₂=15-120.

5. The preparation method according to claim 1, wherein the temperature of crystallization is 120-170° C., and the time of crystallization is 12-120 h.

6. The preparation method according to claim 1, wherein the silicon source includes one or more of silica, silicate salt, and silicate ester.

7. The preparation method according to claim 1, wherein the silicon source includes one or more of silica sol, solid silica gel, white carbon black, water glass, and ethyl orthosilicate.

8. The preparation method according to claim 1, wherein the aluminum source includes one or more of meta-aluminate, aluminum sulfate, alumina, and pseudo boehmite.

9. The preparation method according to claim 1, wherein the crystal seed is a molecular sieve having an MWW topology.

10. The preparation method according to claim 1, wherein the crystal seed comprises an MCM-22 molecular sieve and/or an MCM-49 molecular sieve.

11. The preparation method according to claim 1, wherein the alkali source comprises sodium hydroxide and/or potassium hydroxide.

12. The preparation method according to claim 1, comprising:
mixing an aluminum source, water, an alkali source, a first template agent, and a second template agent to obtain an intermediate solution, adding a silicon source and a crystal seed to the intermediate solution and mixing them to form a gel, and subjecting the gel to crystallization to obtain the MWW-structured molecular sieve.

13. An MWW-structured molecular sieve produced by the preparation method according to claim 1.

14. The MWW-structured molecular sieve according to claim 13, wherein the MWW-structured molecular sieve has a specific surface area of 500 m²·g or more.

15. The MWW-structured molecular sieve according to claim 13, wherein the MWW-structured molecular sieve has a micropore specific surface area of 350 m²·g or more and has a mesopore specific surface area of 140 m²·g or more.

16. The MWW-structured molecular sieve according to claim 13, wherein the MWW-structured molecular sieve has a pore volume of 0.60 cm³·g or more.

17. The MWW-structured molecular sieve according to claim 13, wherein the MWW-structured molecular sieve has a micropore volume of 0.16 cm³·g or more.

18. A method of an alkylation reaction, wherein the method comprising: using the MWW-structured molecular sieve according to claim 13 to catalyze the reaction;
the reaction includes an alkylation reaction, an isomerization reaction or a cracking reaction.

19. The preparation method according to claim 6, wherein the silicon source includes one or more of silica sol, solid silica gel, white carbon black, water glass, and ethyl orthosilicate.
